(19) United States
(12) Patent Application Publication
(10) Pub. No.: US 2022/0280808 A1
(43) Pub. Date: Sep. 8, 2022

(54) AUDIBLE TEMPERATURE READOUT APPARATUS FOR USE WITH A PHOTO-THERMAL TARGETED TREATMENT SYSTEM AND ASSOCIATED METHODS

(71) Applicant: Accure Acne LLC, Boulder, CO (US)

(72) Inventors: Henrik Hofvander, Budapest (HU); Michael John Estes, Lafayette, CO (US); Aubrey Jean Eck, Denver, CO (US)

(73) Assignee: Accure Acne LLC, Boulder, CO (US)

(21) Appl. No.: 17/192,055

(22) Filed: Mar. 4, 2021

(51) Int. Cl. A61N 5/06

(52) U.S. Cl. CPC A61N 5/0616; A61N 5/062; A61N 2005/067

(57) ABSTRACT

A method for operating an energy source within an energy-based treatment system for dermatological procedures. The method includes: 1) administering at least a first energy pulse from the energy source at a first parameter setting to a first treatment location; 2) measuring a peak epidermal temperature (PET) at the first treatment location, following administration of the at least one energy pulse; 3) providing an audio output of the PET so measured; and 4) according to the audio output of the PET, determining whether to adjust the energy source from the first parameter setting.

### PRIORITY CLAIM

The present application claims priority to U.S. Provisional Patent Application No. 62/984,938, filed on Mar. 4, 2020, entitled “Audible Temperature Readout Apparatus for Use with a Photo-Thermal Targeted Treatment System and Associated Methods.” The aforementioned application is incorporated herein in their entirety by reference.

### FIELD OF THE INVENTION

The present invention relates to energy-based treatments and, more specifically, systems and methods for improving the safety and efficacy of an energy-based dermatological treatment.

### BACKGROUND OF THE INVENTION

Sebaceous glands and other chromophores embedded in a medium such as the dermis, can be thermally damaged as part of a therapeutic dermatological procedure by heating the chromophore with a targeted light source, such as a laser. However, the application of enough thermal energy to damage the chromophore can also be detrimentally damaging to the surrounding dermis and the overlying epidermis, thus leading to epidermis and dermis damage as well as pain to the patient.

Previous approaches to prevent epidermis and dermis damage, as well as patient pain include:

1. Cooling the epidermis, then applying the photo-thermal treatment;
and

2. Cool the epidermis, also condition (i.e., preheat) the epidermis and dermis in a preheating protocol, then apply photo-thermal treatment in a distinct treatment protocol. In certain instances, the preheating protocol and the treatment protocol are performed by the same laser, although the two protocols involve different laser settings and application protocols, thus leading to further complexity in the treatment protocol and equipment.

For either of these approaches, as well as in many energy-based dermatological procedures, measuring the temperature of the skin surface during the treatment provides valuable information that can be used to adjust the treatment protocol and/or equipment settings in real time. Such temperature-based treatment protocol adjustments can be made by a practitioner, as an example, every few seconds over an hour-long procedure. However, in the current state of the art, such temperature measurements must be visually confirmed by the practitioner, for instance, by viewing a display or a stripchart, forcing the practitioner to take their eyes away from the treatment location. Such shifting in visual attention of the practitioner can lead to errors in the treatment protocol and, in worst case, injury to the patient under treatment.

### SUMMARY OF THE INVENTION

In accordance with the embodiments described herein, there is described a method for operating a light source within a photo-thermal targeted treatment system for targeting a chromophore embedded within a medium. The method includes: 1) administering at least one laser pulse from the light source at a first parameter setting to a first treatment location; 2) measuring a peak epidermal temperature (PET) at the first treatment location, following administration of the at least one laser pulse; 3) providing an audio output of the PET so measured; and 4) according to the audio output of the PET, determining whether to adjust the light source from the first parameter setting.

### BRIEF DESCRIPTION OF THE DRAWINGS

FIG. 1 shows an exemplary photo-thermal targeted treatment system for targeting a target, wherein the target includes specific chromophores embedded in a medium, and heating the target to a sufficiently high temperature so as to damage the target without damaging the surrounding medium. The system can be used, for example, for photo-thermal ablation of sebaceous glands in a targeted fashion, where sebum is the chromophore embedded within the sebaceous gland, while sparing the epidermis and dermis surrounding the target sebaceous glands.

FIG. 2 is a flow diagram illustrating an exemplary method for operating a light source within a photo-thermal targeted treatment system

### DETAILED DESCRIPTION OF EMBODIMENTS OF THE INVENTION

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items, and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

When performing a dermatological treatment, it is preferable that the practitioner keeps his/her eyes continuously on the current treatment location. The reasons include:

1. Safety
A. The practitioner can ensure that the system is performing correctly by viewing e.g. aiming a beam that is in the visual range
B. The practitioner can ensure that there are no adverse events, e.g. blisters on the skin surface
C. The practitioner can ensure that no treatment areas are being overlapped, ie the same area is being treated twice. Anytime the practitioner looks away from the treatment area there is a risk the s/he loses track of where the last treatment took place and where to go to next.

2. Efficacy: The practitioner can ensure that no treatment areas are being skipped. For instance, keeping the practitioner's attention at the treatment location makes it less likely that the practitioner will lose track of where the next treatment location is if s/he is following a grid pattern.

3. Ergonomics: Having to constantly swivel his/her head to look at a screen is not a good ergonomic setup.

4. Economics: Currently, an assistant is required in the treatment room to call out temperatures, even if the temperature is displayed on the treatment system user interface. Having an assistant attend the procedure, even if the assistant is not required in the use of the treatment system, adds to the cost of operating the treatment system.

As discussed above, peak epidermal temperature (PET) measurements of the treatment area during application of the treatment protocol can assist in the prevention of patient injury while improving efficacy of the treatment. While the PET reading could be displayed on the treatment system user interface, such as on a screen or strip chart, even such a visual display requires the practitioner to momentarily take his/her eyes away from the treatment area.

Instead, the embodiment described in the present disclosure includes an audible readout of PET via an earpiece, one or more headphones, or a speaker such that the practitioner can utilize the PET information in making adjustments to the treatment protocol and treatment system settings (e.g., light source parameter settings) while keeping the practitioner free to keep his/her eyes at the treatment location. While the audible readout can also be made through a speaker, for patient comfort it can be preferred that the audible readouts are just heard by the practitioner.

In laser treatment of acne, the operating thermal range is generally bound on the upper end at the epidermis and dermis damage threshold temperature of approximately 55° C., and at the lower end by the temperature required to bring the sebaceous gland to its damage threshold temperature of approximately 55° C. Based on clinical data, the operating temperature range for acne treatment expressed in terminal skin surface temperature is approx. 40° C. to 55° C., as an example. At skin surface temperatures below 40° C., it has been determined that there is no damage to the sebaceous gland. When the skin surface temperature is between 40° C. and 55° C., there are varying degrees of sebaceous gland damage, with no epidermal damage. Above 55° C., there is epidermal damage in addition to damage to the sebaceous gland. By adjusting the treatment system, such as the light source parameter settings, in real time during treatment, the practitioner can avoid damaging the epidermis and dermis surrounding the treatment area, while effectively administering the treatment protocol.

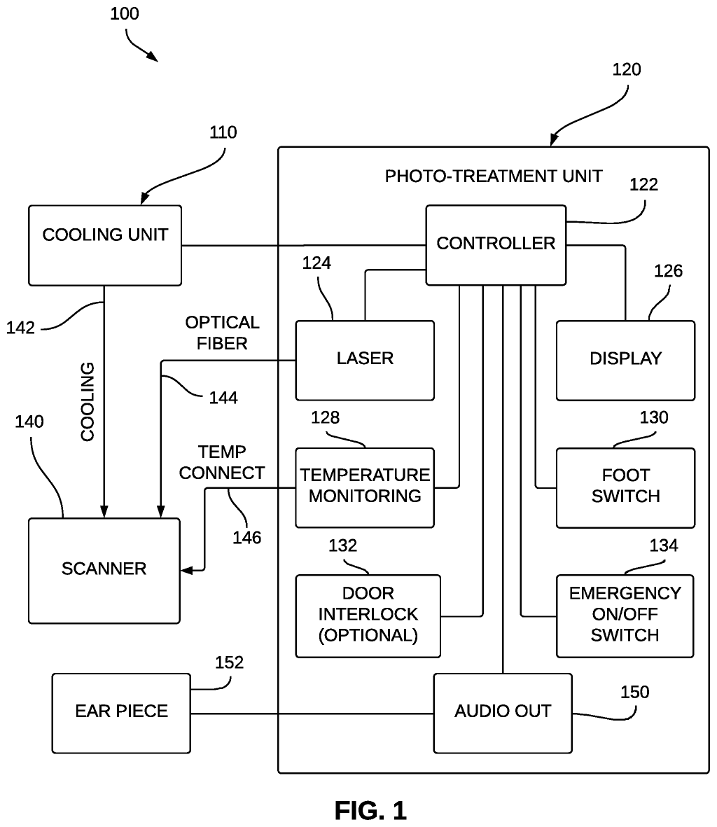

FIG. 1 shows an exemplary photo-thermal targeted treatment system for targeting a target, wherein the target includes specific chromophores embedded in a medium, and heating the target to a sufficiently high temperature so as to damage the target without damaging the surrounding medium. The system can be used, for example, for photo-thermal ablation of sebaceous glands in a targeted fashion, where sebum is the chromophore embedded within the sebaceous gland, while sparing the epidermis and dermis surrounding the target sebaceous glands.

Still referring to FIG. 1, a photo-thermal targeted treatment system 100 includes a cooling unit 110 and a photo-treatment unit 120. Cooling unit 110 provides a cooling mechanism for a cooling effect to a treatment area, namely the outer skin layer overlying the target sebaceous gland. The cooling effect may be accomplished by contacting the skin with a cold solid medium to conduct heat away from the treatment area or by direct air cooling to convect heat away from the treatment area. Cooling unit 110 is connected with a controller 122 within photo-treatment unit 120. It is noted that, while controller 122 is shown to be contained within photo-treatment unit 120 in FIG. 1, it is possible for the controller to be located outside of both cooling unit 110 and photo treatment unit 122, or even within cooling unit 110.

Controller 122 further controls other components within photo-treatment unit 120, such as a laser 124, a display 126, a temperature monitoring unit, a foot switch 130, a door interlock 132, and an emergency on/off switch. Laser 124 provides the laser power for the photo-treatment protocol, and controller 122 regulates the specific settings for the laser, such as the output power and pulse time settings. Laser 124 can be a single laser or a combination of two or more lasers. If more than one laser is used, the laser outputs are combined optically to function as one more powerful laser. Display 126 can include information such as the operating conditions of cooling unit 110, laser 124, and other system status. Temperature monitoring unit 128 is used to monitor the temperature of the skin surface in the treatment area, for example, and the measured skin surface temperature at the treatment area is used by controller 122 to adjust the photo-treatment protocol. Controller 122 also interfaces with footswitch 130 for remotely turning on or off laser 124 and/or cooling unit 110. Additionally, door interlock 132 can be used as an additional safety measure such that, when the door to the treatment room is ajar, door interlock 132 detects the condition and instructs controller 122 to not allow photo-treatment unit 120, or at least laser 124, to operate. Furthermore, emergency on/off switch 134 can be provided to quickly shut down photo-thermal targeted treatment system 100 in case of an emergency. In another modification, additional photodiodes or other sensors can be added to monitor the power level of the energy emitted from laser 124.

Continuing to refer to FIG. 1, photo-thermal targeted treatment system 100 further includes a scanner 140, which is the portion of the device handheld by the user in applying the treatment protocol to the subject. Scanner 140 can be formed, for example, in a gun-like or stick-like shape for ease of handling by the user. Scanner 140 is connected with cooling unit 110 via a cooling connection 142, such that the cooling protocol can be applied using scanner 140. Additionally, the output from laser 124 is connected with scanner 140 via an optical fiber delivery 144, such that the photo-treatment protocol can be applied using scanner 140. Scanner 140 is connected via a temperature connection 146 to temperature monitoring unit 128, so as to feedback the skin temperature at the treatment area, for example, to controller 122.

Additionally, photo-treatment unit 120 further includes an audio out circuitry 150 for providing an audio output, such as a PET reading as recorded at temperature monitoring unit 128. Audio out circuitry 150 provides a signal to, for example, an ear piece 152 through a wired or wireless connection such that the practitioner using the system can listen to the audio output. Ear piece 152 can be replaced, for instance, by a speaker system or other audio communication means. Audio out circuitry can also convey other information such as the status of the photo-treatment unit, any emergency warnings, or other messages to be conveyed to the user of photo-thermal targeted treatment system 100. The ear piece 152, speaker, headphone, or other audio output device may include user input components such as volume controls, repeat message control, or a microphone that can be used to provide user input to the audio out circuitry 150 and/or the controller 122.

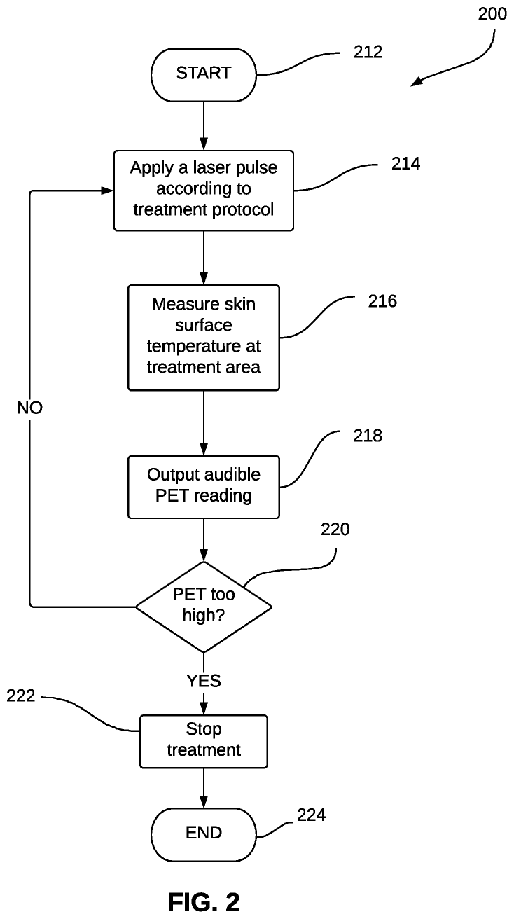

FIG. 2 is a flow diagram illustrating an exemplary method for operating a light source within a photo-thermal targeted treatment system. A process 200 begins with a start step 212 to initialize the treatment protocol. A laser pulse is applied to the treatment area according to the treatment protocol in a step 214. The treatment protocol may determine particular characteristics of the laser pulse, such as power, pulse width, and duration. The real-time skin surface temperature (i.e., PET) at the treatment area is measured in a step 216. An audible PET reading is output to the practitioner in a step 218. The audible reading may be a vocalization of a number by a pre-recorded or digital voice, and the temperature measurement may be read out in a selected temperature unit scale (e.g., Celcius, Fahrenheit, or Kelvin). Alternatively or additionally, warning signals such as beeps, melodic tones, or other audio signals can be used to represent warnings, system status, or other important notifications. Based on the audible reading of the PET temperature, the practitioner determines, in a decision 220, whether the PET is too high (i.e., whether the measured PET is close to the damage threshold for the epidermis and/dermis at the treatment area, or whether the PET is within a therapeutic temperature range). If the answer to decision 220 is NO, the PET is not too high or is not yet within the therapeutic temperature range, then the process returns to step 214 to apply another laser pulse to the treatment area. The second (or subsequent) laser pulse may be applied using the same or different settings as used during application of the first laser pulse. In some embodiments, the second laser pulse is adjusted based on the PET measurement or based on the audio output of the PET measurement. If the answer to decision 220 is YES, the PET is too high (or is indicated as being within the therapeutic temperature range) and approaching the damage threshold for the treatment area, then the practitioner stops the treatment in a step 222, and the process is ended in an end step 224.

In some embodiments, even if the answer to decision 220 is NO, the treatment method may be stopped at step 222 if the user notices damage or abnormal skin reactions. For example, a second decision step (not shown) may be included where, from a NO answer at decision 220, the process asks whether a user has observed any concerning skin abnormalities in response to the laser treatment. If the answer is YES, the process may proceed to finish step 222; if the answer is NO the process may proceed to step 214 where additional treatment pulses are determined and applied as normal. As discussed above, the audible PET measurement readout feature of the system 100 allows the user to constantly observe and evaluate the appearance of skin within the treatment area without requiring the user to look away during the treatment to obtain information relevant to the treatment. The treatment process may take advantage of the user's improved attention to skin appearance by providing an additional safety check that takes into account the user's professional assessment of the skin based on constant observation. For example, during the treatment process, the user may notice a small blister forming on the patient's skin and would be able to quickly assess and halt treatment as needed using the second decision block. Thus, the constant treatment area observation facilitated by audio readouts can be accommodated in the treatment process to advantageously provide an extra layer of user input for improved patient safety.

Once the process ends at step 222, the user may select a second treatment area different from the first treatment area and may repeat the process 200 again from the beginning on a new area of skin. The second treatment area may be selected such that bounds of the first and second treatment areas do not overlap, but no untreated areas are left in between, so that the patient's skin is evenly treated. The audible readout feature included in the system 100 allows for the user to keep eyes on the patient at all times so as to facilitate this precise alignment between treatment areas.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

For example, embodiments such as the below are contemplated:

1. A method for operating an energy-based treatment system for dermatological procedures. The method includes: 1) administering at least one energy pulse at a first parameter setting to a first treatment location; 2) measuring a peak epidermal temperature (PET) at the first treatment location, following administration of the at least energy pulse; 3) providing an audio output of the PET so measured; and 4) according to the audio output of the PET, determining whether to adjust the energy source from the first parameter setting.

The audio output can also be used for additional functionality to perform tasks that would otherwise force the practitioner to take his/her eyes off of the treatment area. For example, the system can be set to issue audible warning messages (e.g., “PET is approaching the preset threshold level”), error messages (e.g., “Something has gone wrong—system is shutting down”), and timing notifications (e.g., “Treatment time thus far—30 minutes”).

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the claimed invention

What is claimed is:

1. A method for operating an energy source within an energy-based treatment system for dermatological procedures, the method comprising:
administering to a first treatment location a first energy pulse from the energy source at a first parameter setting;
measuring a peak epidermal temperature (PET) at the first treatment location following administration of the first energy pulse;
providing an audio output of the PET; and
based on the audio output of the PET, determining whether the PET is below or within a therapeutic threshold temperature range.

2. The method of claim 1, wherein if the PET is below a therapeutic threshold temperature range, a second parameter setting for the energy source is determined for a second energy pulse at the first treatment location based on the audio output of the PET.

3. The method of claim 1, wherein if the PET is below a therapeutic threshold temperature range, a user indicates whether abnormalities are present in the treatment area as a result of the first energy pulse.

4. The method of claim 3, wherein the user indicates there are no abnormalities and the second parameter setting for the energy source is determined for the second energy pulse at the first treatment location based on the audio output of the PET.

5. The method of claim 3, wherein the user indicates there are abnormalities and treatment ends at a stop step.

6. The method of claim 1, wherein if the PET is within the therapeutic threshold temperature range, the first parameter setting of the energy source is adjusted to reduce an amount of energy released by the energy source prior to administering a second energy pulse.

7. The method of claim 1, wherein if the PET is within the therapeutic threshold temperature range, the treatment ends at the stop step.

8. The method of claim 7, wherein the treatment proceeds to the stop step automatically based on the PET being within the therapeutic threshold temperature range.

9. The method of claim 8, wherein the system provides a notification to the user that the treatment automatically proceeded to the stop step.

10. The method of claim 1, wherein the audio output comprises a verbal reading of the PET temperature measurement.

11. The method of claim 1, wherein the audio output comprises a non-verbal audio signal.

12. A system for performing a dermatological procedure, the system comprising:
a photo-treatment unit comprising:
a controller;
an energy source coupled with the controller and configured to receive energy source instructions from the controller;
a temperature monitoring unit coupled with the controller and configured to receive temperature monitoring instructions from and send temperature measurements to the controller; and
an audio output unit coupled with the controller and configured to receive audio instructions from the controller;
a scanner coupled with the energy source and configured to receive light from the energy source; and
an audio output device configured to receive data from the audio output unit and configured to emit a sound.

13. The system of claim 12, wherein the controller is configured to adjust parameters of the energy source based on the temperature measurements.

14. The system of claim 12, wherein the audio output device comprises at least one selected from a group consisting of a speaker, an ear piece, and a headphone.

15. The system of claim 12, wherein the speaker is configured to receive data wirelessly from the audio output unit.

16. The system of claim 12, wherein the sound comprises a verbal reading of at least one temperature measurement.

17. The system of claim 16, wherein the at least one temperature measurement comprises a peak epidermal temperature (PET) of skin within a treatment area.

18. The system of claim 12, wherein the sound comprises a verbal reading of a treatment duration.

19. The system of claim 12, wherein the sound comprises a non-verbal audio signal.
